(19) United States
(12) Patent Application Publication
(10) Pub. No.: US 2025/0281071 A1
(43) Pub. Date: Sep. 11, 2025

(54) AUDITORY ENHANCEMENT METHOD, AUDITORY ENHANCEMENT SYSTEM, READABLE STORAGE MEDIUM, AND COMPUTER DEVICE

(71) Applicant: SHENZHEN QIAN HAI WOER TECHNOLOGY LIMITED, SHENZHEN (CN)

(72) Inventors: QI LIN, SHENZHEN (CN); ZHAOBAI LUO, SHENZHEN (CN)

(21) Appl. No.: 19/215,285

(22) Filed: May 21, 2025

Related U.S. Application Data
(63) Continuation of application No. PCT/CN2023/080190, filed on Mar. 8, 2023.

(30) Foreign Application Priority Data
Nov. 21, 2022 (CN) 202211456189.6

(51) Int. Cl. A61B 5/12; H04R 1/10; H04R 3/04

(52) U.S. Cl. CPC A61B 5/123; H04R 1/1041; H04R 3/04; H04R 2430/01

(57) ABSTRACT

An auditory enhancement method, an auditory enhancement system, a readable storage medium, and a computer device are provided. The auditory enhancement method includes extracting frequency points in initial audio data and fusing each of the frequency points with a test short-time audio having different intensities to obtain test audio data; creating an auditory test environment for a user, playing the test audio data to the user in the auditory test environment, and collecting test results fed back by the user; obtaining identity information of the user, and inputting the identity information and the test results into an auditory test mathematical model to obtain auditory compensation values corresponding to the frequency points in the test audio data; and generating auditory gain values corresponding to the auditory compensation values according to the auditory compensation values, and enabling the user to enhance auditory sensitivity according to the auditory gain values.

### TECHNICAL FIELD

The present disclosure relates to a technical field of speech digital signal processing, and in particular to an auditory enhancement method, an auditory enhancement system, a readable storage medium, and a computer device.

### BACKGROUND

With rapid development of science and technology and improvement of people's living standards, people's requirements for entertainment are getting higher and higher. Many people wear earphones to enjoy music, so people's requirements for sound quality of the earphones are getting higher and higher.

In the prior art, a sound with a frequency range of 6400-12800 Hz is defined as treble, and a sound with a frequency range of 20-800 Hz is defined as bass. Because different people have different sensitivities to sounds in different frequency bands, some people are sensitive to treble but not to bass, while some people are not sensitive to treble but sensitive to bass. In view of such situation, in order to hear a sound of a certain frequency band more easily, people usually turn up a volume of the earphones. However, turning up the volume of the earphones makes it easier to cover up details of the sound, reduce sound quality, and affect people's hearing. In addition, listening to loud sounds for a long time may affect the comfort of human ears.

### SUMMARY

In order to solve problems in the prior art, the present disclosure provides an auditory enhancement method, an auditory enhancement system, a readable storage medium, and a computer device.

The present disclosure provides the auditory enhancement method. The auditory enhancement method is applied to a pair of earphones, and the pair of earphones is communicated with a user terminal. The auditory enhancement method includes steps:
extracting frequency points in initial audio data by a predetermined extraction rule, and fusing each of the frequency points with a test short-time audio with different intensities to obtain test audio data;
creating an auditory test environment for a user, playing the test audio data to the user in the auditory test environment, and collecting test results fed back by the user based on the test audio data, where the test results are respectively corresponding to the frequency points;
obtaining identity information of the user, and inputting the identity information and the test results into an auditory test mathematical model to obtain auditory compensation values one-to-one corresponding to the frequency points in the test audio data; and
generating auditory gain values corresponding to the auditory compensation values according to the auditory compensation values, and enabling the user to enhance auditory sensitivity according to the auditory gain values.

Furthermore, the step of creating the auditory test environment for the user comprises steps:
obtaining environmental audio data of a current environment, and determining whether the environmental audio data exceeds an environmental audio threshold; and
creating the auditory test environment for the user based on the current environment when the environmental audio data does not exceed the environmental audio threshold.

Furthermore, the identity information comprises age information and gender information. The step of inputting the identity information and the test results into the auditory test mathematical model to obtain the auditory compensation values one-to-one corresponding to the frequency points in the test audio data comprises steps:
performing difficulty analysis on the test short-time audio in each of the frequency points based on a difficulty database to obtain a difficulty coefficient corresponding to the test short-time audio at each of the frequency points;
respectively calculating an age coefficient and a gender coefficient according to the age information and the gender information; and
respectively calculating a weight value of each difficulty coefficient, a weight value of the age coefficient, a weight value of the gender coefficient, and an average value of the test results, and calculating the auditory compensation values respectively corresponding to the frequency points according to the weight value of each difficulty coefficient, the weight value of the age coefficient, the weight value of the gender coefficient, the average value of the test results, and the auditory test mathematical model

Furthermore, the step of generating the auditory gain values corresponding to the auditory compensation values according to the auditory compensation values, and enabling the user to enhance the auditory sensitivity according to the auditory gain values comprises steps:
mapping the auditory compensation values to the user terminal to obtain terminal mapping values; and
generating the auditory gain values based on the terminal mapping values to enable the user to enhance the auditory sensitivity according to the auditory gain values.

Furthermore, expression formulas of each of the auditory compensation values are:

$$Tave = \frac{1}{N} * {\sum }_{i = 0}^{N - 1} ( {T}_{i} ) ;$$

$${Tave}_{W} = \frac{Tave}{{Tave}_{S}} ; and$$

$$S = B * ( Wt * D * {T}_{i} + Wa * A * {Tave}_{W} + Wg * G * {Tave}_{W} ) .$$

Tave represents the test average value of the test results of the frequency points, N represents the number of the frequency points, Tᵢ represents a test result of each of the frequency points, S represents a corresponding auditory compensation value, B represents an auditory compensation reference value, Wt represents the weight value of a sample average value of the frequency points, D represents a corresponding difficulty coefficient, A represents the age coefficient, Wa represents the weight value of the age coefficient, Taveₛ represents the sample average value of the frequency points, Tave_{w} is a ratio of the test average value of the frequency points to the sample average value of the frequency points and represents a weight value of the test average value, Wa represents the weight value of the gender coefficient, and G represents the gender coefficient.

The present disclosure further provides the auditory enhancement system. The auditory enhancement system comprises a test audio construction module, a test environment creation module, a compensation value calculation module, and a gain value calculation module.

The test audio construction module is configured to extract frequency points in initial audio data by a predetermined extraction rule and fuse a test short-time audio having different intensities to each of the frequency points to obtain test audio data.

The test environment creation module is configured to create an auditory test environment for a user, play the test audio data to the user in the auditory test environment, and collect test results fed back by the user based on the test audio data.

The compensation value calculation module is configured to obtain identity information of the user and input the identity information and the test results into an auditory test mathematical model to obtain auditory compensation values one-to-one corresponding to the frequency points in the test audio data.

The gain value calculation module is configured to generate auditory gain values corresponding to the auditory compensation values according to the auditory compensation values, so that the user enhances auditory sensitivity according to the auditory gain values.

Furthermore, the test environment creation module comprises an environmental audio acquisition unit and a test environment creation unit.

The environmental audio acquisition unit is configured to obtain environmental audio data of a current environment and determine whether the environmental audio data exceeds an environmental audio threshold.

The test environment creation unit is configured to create the auditory test environment for the user based on the current environment when the environmental audio data does not exceed the environmental audio threshold.

Furthermore, the identity information comprises age information and gender information. The compensation value calculation module comprises a difficulty analysis unit, a coefficient calculation unit, and a compensation value calculation unit.

The difficulty analysis unit is configured to perform difficulty analysis on the test short-time audio in each of the frequency points based on a difficulty database to obtain a difficulty coefficient corresponding to the test short-time audio in each of the frequency points.

The coefficient calculation unit is configured to respectively calculate an age coefficient and a gender coefficient according to the age information and the gender information.

The compensation value calculation unit is configured to respectively calculate a weight value of each difficulty coefficient, a weight value of the age coefficient, a weight value of the gender coefficient, and an average value of the test results, and calculate the auditory compensation values respectively corresponding to the frequency points according to the weight value of each difficulty coefficient, the weight value of the age coefficient, the weight value of the gender coefficient, the average value of the test results, and the auditory test mathematical model.

The present disclosure further provides the readable storage medium. The readable storage medium comprises a computer program stored thereon. When the computer program is executed by a processor, the auditory enhancement method mentioned above.

The present disclosure further provides the computer device. The computer device comprises a memory, a processor, and a computer program stored in the memory and executable on the processor. The processor is configured to execute a computer program to implement the auditory enhancement method mentioned above.

In the auditory enhancement method, the auditory enhancement system, the readable storage medium, and the computer device of the present disclosure, the frequency points are extracted, the test short-time audio with different intensities is fused to each of the frequency points, the auditory compensation values corresponding to the frequency points are obtained according to the test results fed back by the user and the identity information of the user, and the auditory gain values are generated corresponding to the auditory compensation values. Therefore, the user is enabled to enhance the auditory sensitivity according to the auditory gain values, thereby enhancing perception intensity of the user on a sound with insensitive frequency bands without changing a sound volume of sound. Thus, the user is allowed to appreciate music in a wide frequency range.

### BRIEF DESCRIPTION OF DRAWINGS

FIG. 1 is a flow chart of an auditory enhancement method according to a first embodiment of the present disclosure.

FIG. 2 is a flow chart of step S102 shown in FIG. 1.

FIG. 3 is a flow chart of step S103 shown in FIG. 1.

FIG. 4 is a flow chart of step S104 shown in FIG. 1.

FIG. 5 is a block diagram of an auditory enhancement system according to a second embodiment of the present disclosure.

FIG. 6 is a block diagram of a computer device according to a third embodiment of the present disclosure.

The following specific embodiments of the present disclosure are further illustrated in conjunction with the drawings.

### DETAILED DESCRIPTION

For ease of understanding the present disclosure, the present disclosure is described fully hereinafter with reference to the accompanying drawings. Several embodiments of the present disclosure are given in the accompanying drawings. However, the present disclosure may be implemented in many different forms and is not limited to the embodiments described herein. On the contrary, a purpose of providing these embodiments is to make the disclosure of the present disclosure more thorough and comprehensive.

It should be noted that when an element is referred to as being “fixed to” another element, it may be directly fixed to another element or indirectly fixed to another element through intervening elements. When the element is considered to be “connected” to another element, it may be directly connected to another element or intervening elements may be present at the same time. The terms “vertical”, “horizontal”, “left”, “right”, and the like, as used herein, are for illustrative purposes only.

Unless otherwise defined, all technical and scientific terms used herein have the same meaning as commonly understood by those skilled in the art of the present disclosure. The terms used in the specification of the present disclosure are only for the purpose of describing specific embodiments, and are not intended to limit the present disclosure. As used herein, the term “and/of” includes all combinations including one or more of associated listed items.

### Embodiment 1

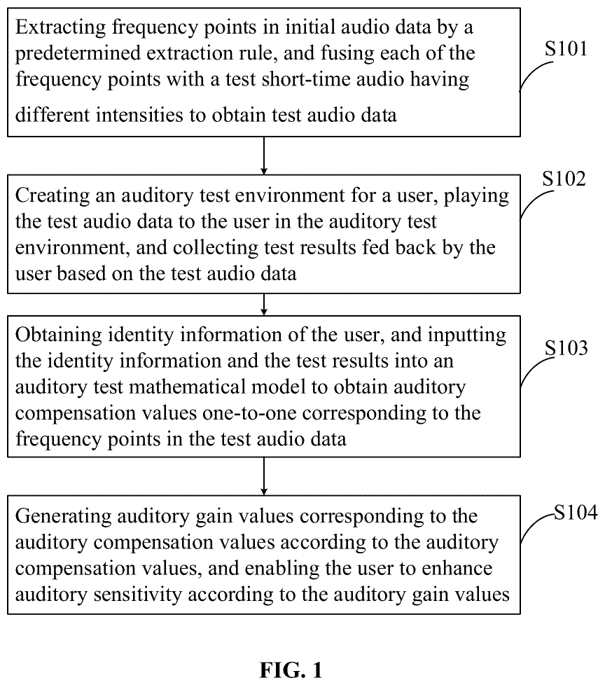

As shown in FIG. 1, the embodiment of the present disclosure provides an auditory enhancement method. The auditory enhancement method includes steps S101-S104.

The step S101 comprises extracting frequency points in initial audio data by a predetermined extraction rule, and fusing a test short-time audio having different intensities to each of the frequency points to obtain test audio data.

The auditory enhancement method in the embodiment is applied to a sound-producing device with higher sound quality, such as a pair of earphones or a speaker. Specifically, the initial audio data is obtained. The initial audio data is audio data configured to adjust perceived intensity of a user on different frequency bands before an auditory test. The initial audio data comprises audio information of different frequency bands. From an entire sound spectrum of the initial audio data, the frequency points are extracted according to predetermined sound frequencies. In the embodiment, the predetermined sound frequencies comprise 10 frequency points, which are respectively 20 Hz, 50 Hz, 100 Hz, 200 Hz, 400 Hz, 800 Hz, 1600 Hz, 3200 Hz, 6400 Hz, and 12800 Hz.

After obtaining the 10 frequency points, a test short-time audio having different intensities is allocated to each of the frequency points, and the test short-time audio is fused with each of the frequency points of the entire sound spectrum to obtain the test audio data. In the embodiment, the test short-time audio has four sound intensities, and the four sound intensities are 67 dB, 60 dB, 55 dB, and 50 dB respectively. The short-time audio is an audio with a duration not less than 1 second and is distinguished from the entire sound spectrum, such as a sound of beeps.

The step S102 comprises creating an auditory test environment for a user, playing the test audio data to the user in the auditory test environment, and collecting test results fed back by the user based on the test audio data.

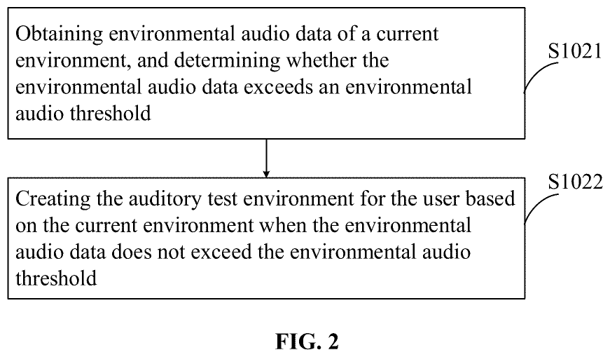

Furthermore, as shown in FIG. 2, the step S102 further comprises steps S1021-S2022.

The step S1021 comprises obtaining environmental audio data of a current environment, and determining whether the environmental audio data exceeds an environmental audio threshold.

The step S1022 comprises creating the auditory test environment for the user based on the current environment when the environmental audio data does not exceed the environmental audio threshold.

Specifically, when the user uses a terminal device (the pair of earphones, a mobile phone, a speaker, a tablet, a laptop, or other devices with microphone and speaker functions, in the embodiment, the pair of earphones and the mobile phone are taken as examples) to perform the auditory test, the pair of earphones is communicated with the mobile phone, and a corresponding application (APP) installed on the mobile phone is opened at the same time. The environmental audio of the current environment is collected through the pair of earphones, and the environmental audio is converted into a decibel value in the corresponding APP to obtain the environmental audio data. The environmental audio data of the current environment is obtained, and an environmental sound intensity of the current environment is determined based on the environmental audio data. When the environmental audio data is greater than the environmental audio threshold (in the embodiment, the environmental audio threshold is 30 dB), it means that a sound intensity of the environmental audio data is too loud. If performing the auditory test in the current environment, the test results are affected, so the auditory test is necessary to be performed in another environment. When the environmental audio data is not greater than the environmental audio threshold, it means that the sound intensity of the environmental audio data is low, which meets a test environment requirement of the auditory test, and the auditory test environment for the user is created based on the current environment.

In the embodiment, when the environmental audio data is greater than the environmental audio threshold, the APP outputs a prompt signal to the user via the pair of earphones, a corresponding noise reduction mode signal of the pair of earphones is triggered at the same time, and a buffer pulse is output by the pair of earphones in reverse to reduce the sound intensity of the environmental audio data, so that the environmental audio data meets the test environment requirement of the auditory test.

After obtaining the environmental audio data that meets the test environment requirement, the current environment corresponding to the environmental audio data is served as the auditory test environment.

Furthermore, after the auditory test environment for the use is created, the test audio data is played to the user in the auditory test environment. When playing the test audio data, and when the user hears the test short-time audio at any one of the frequency points (i.e., the beeps with different sound intensities of 67 dB, 60 dB, 55 dB, and 50 dB), the user feeds back a signal indicating that the test short-time audio has been heard. When the user does not hear the test short-time audio, a signal indicating that the test short-time audio has not been heard by the user is transmitted. During audio playback, the signals corresponding to each of the frequency bands are collected in real time, and the sound intensities corresponding to the signals is recorded to generate the test results corresponding to the frequency points.

The step S103 comprises obtaining identity information of the user, and inputting the identity information and the test results into an auditory test mathematical model to obtain auditory compensation values one-to-one corresponding to the frequency points in the test audio data.

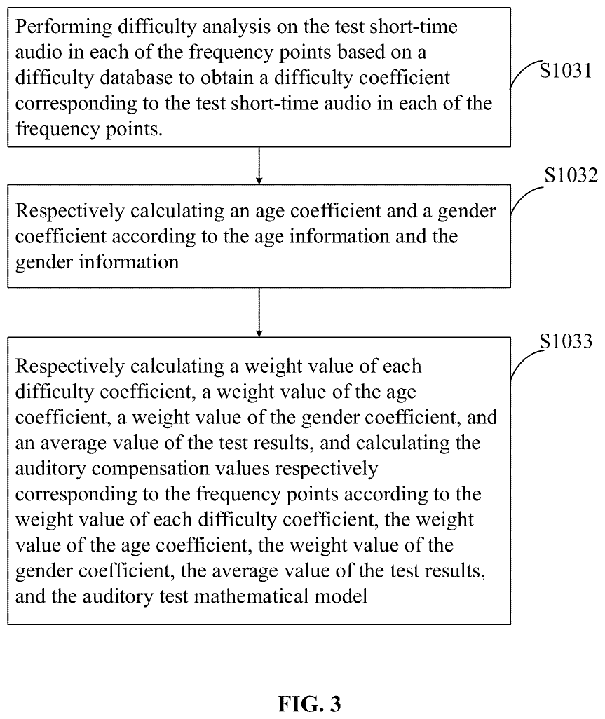

Furthermore, the identity information comprises age information and gender information. As shown in FIG. 3, the step S103 comprises steps S1031-S1033.

The step S1031 comprises performing difficulty analysis on the test short-time audio at each of the frequency points based on a difficulty database to obtain a difficulty coefficient corresponding to the test short-time audio at each of the frequency points.

The step S1032 comprises respectively calculating an age coefficient and a gender coefficient according to the age information and the gender information.

The step S1033 comprises respectively calculating a weight value of each difficulty coefficient, a weight value of the age coefficient, a weight value of the gender coefficient, and an average value of the test results, and calculating the auditory compensation values respectively corresponding to the frequency points according to the weight value of each difficulty coefficient, the weight value of the age coefficient, the weight value of the gender coefficient, the average value of the test results, and the auditory test mathematical model.

In one specific embodiment, registration information of the user is obtained when the user logs in the APP, and the registration information comprises age, gender and other identity information of the user. Difficulty coefficients are constructed according to the auditory sensitivity of the human body to different sound frequencies, and the difficulty database is constructed according to the difficulty coefficients corresponding to different sound frequencies and the sound intensities. For instance, Table 1 is a mapping table showing some examples of relationships between the sound frequencies and the difficulty coefficients.

TABLE 1

| | Sound frequency (Hz) | | | | | | | | | |
|---|---|---|---|---|---|---|---|---|---|---|
| | 20 | 50 | 100 | 200 | 400 | 800 | 1600 | 3200 | 6400 | 12800 |
| Difficulty | 1.0 | 0.9 | 0.7 | 0.5 | 0.3 | 0.15 | 0.1 | 0.15 | 0.4 | 0.9 |
| coefficient | | | | | | | | | | |
| (D) | | | | | | | | | | |

In Table 1, the maximum difficulty coefficient is 1 and the minimum difficulty coefficient is 0. The greater the difficulty coefficient, the more difficult it is to capture a corresponding sound frequency. Specifically, an ultra-low sound with the sound frequency of 20 Hz is the most difficult to capture, so a difficulty coefficient thereof is set to 1.0. Similarly, a low sound frequency of 50 Hz and a high sound frequency of 12800 Hz are relatively difficult to capture, so difficulty coefficients thereof are set to 0.9. The sound frequencies of 800 Hz, 1600 Hz, and 3200 Hz are the most sensitive sound frequencies to the human ear, so difficulty coefficients thereof are relatively small.

Furthermore, auditory sensitivities of users of different ages to different sound frequencies are collected, and collected data is constructed into an age information database. The age information in the identity information of the user is input into the age information database to obtain the age coefficient corresponding to the user. Table 2 is a mapping table showing some examples of relationships between the age information and the age coefficient.

TABLE 2

| | Age information | | | |
|---|---|---|---|---|
| | age < 20 | 20 ≤ age < 30 | 30 ≤ age < 40 | age ≥ 40 |
| Age coefficient | 0 | 0.1 | 0.3 | 0.45 |
| (A) | | | | |

It can be seen from Table 2 that users under the age of 20 have a higher auditory sensitivity, so no compensation is needed and a corresponding age coefficient is 0. When the users are within the age of 20-30, the auditory sensitivity of the human ear tends to decrease, so corresponding age coefficients are set to 0.1 and 0.3 respectively. When the age of the users reaches 40, the auditory ability of the human ear is solidified, so a corresponding age coefficient is set to 0.45.

Furthermore, by randomly selecting people of the same age and performing the sound sensitivity test on them, a mapping table of gender to gender coefficients is obtained.

TABLE 3

| Gender | Male | Female |
|---|---|---|
| Gender coefficient | 0.2 | 0.1 |
| (G) | | |

Through random surveys of people of the same age, it is found that males are generally less sensitive to sounds of high-frequency and sounds of ultra-low-frequency than the females. Therefore, the gender coefficient of a male is 0.2 and the gender coefficient of a female is 0.1.

In the embodiment, the test results of different frequency points at different sound intensities are matched with the test results according to a sound intensity test table (Table 4), where the sound intensity test table is as follows:

TABLE 4

| Sound intensity (dB) | 67 | 60 | 55 | 50 | Test result (T) |
|---|---|---|---|---|---|
| Tests sample | 1 | 1 | 1 | 1 | 0.0 |
| | 1 | 1 | 1 | 0 | 0.25 |
| | 1 | 1 | 0 | X | 0.5 |
| | 1 | 0 | X | X | 0.75 |
| | 0 | X | X | X | 1.0 |

In Table 4, 1 indicates that a beep corresponding to a specific sound intensity sound is heard by the user, 0 indicates that the beep corresponding to the specific sound intensity is not heard by the user, and X indicates that a result thereof is null.

After obtaining each difficulty coefficient, the age coefficient, the gender coefficient, and the test results, the test average value of the test results of the frequency points is calculated according to the following formula:

$$Tave = \frac{1}{N} * {\sum }_{i = 0}^{N - 1} ( {T}_{i} ) .$$

Tave represents the test average value of the test results of the frequency points, N represents the number of the frequency points, and Tᵢ represents a test result of each of the frequency points.

For example: when a test result of a first frequency point with the sound frequency 20 Hz at four sound intensities of 67 dB, 60 dB, 55 dB, and 50 dB is 1, a test result of a second frequency point with the sound frequency of 50 Hz is 0.9, a test result of a third frequency point with the sound frequency of 100 Hz is 0.5, a test result of the fourth frequency point with the sound frequency of 200 Hz is 0.5, a test result of a fifth frequency point with the sound frequency of 400 Hz is 0.25, a test result of a sixth frequency point with the sound frequency of 800 Hz is 0, a test result of a seventh frequency point with a sound frequency of 1600 Hz is 0, a test result of an eighth frequency point with the sound frequency of 3200 Hz is 0, a test result of a ninth frequency point with the sound frequency of 6400 Hz is 0.25, and a test result of a tenth frequency point with the sound frequency of 12800 Hz is 0.5, then the test average value Tave of the ten frequency points is 0.375.

The sample average value Taveₛ is calculated according to the test results of the sample data in Table 4. For instance, the test results in Table 4 are 0, 0.25, 0.5, 0.75, and 1, and the sample average value Taveₛ is 0.5. That is, the sample average value is a constant of 0.5.

Furthermore, weight values are allocated to the test average value Tave, sample average value Taveₛ, the age coefficient A, and the gender coefficient G according to a weight allocation rule. Specifically, the weight value Tave_{w} of the test average value Tave is 0.75, the weight value Wt of the sample average value Taveₛ is 0.7, the weight value Wa of the age coefficient A is 0.2, and the weight value Wg of the gender coefficient G is 0.1. The weight values are obtained according to corresponding weight formulas and corresponding weight databases. Of course, the weight values are allowed to be determined by the user.

The weight value of each difficulty coefficient, the weight value of the age coefficient, the weight value of the gender coefficient, and test results obtained above are input into the auditory test mathematical model to calculate the auditory compensation value corresponding to each frequency point:

$${Tave}_{W} = \frac{Tave}{{Tave}_{S}} ; and$$

$$S = B * ( Wt * D * {T}_{i} + Wa * A * {Tave}_{W} + Wg * G * {Tave}_{W} ) .$$

Tave represents the test average value of the test results of the frequency points, N represents the number of the frequency points, Tᵢ represents a test result of each of the frequency points, S represents a corresponding auditory compensation value, B represents an auditory compensation reference value, Wt represents the weight value of a sample average value of the frequency points, D represents a corresponding difficulty coefficient, A represents the age coefficient, Wa represents the weight value of the age coefficient, Taveₛ represents the sample average value of the frequency points, Tave_{w} represents a weight value of the test average value, Wa represents the weight value of the gender coefficient, and G represents the gender coefficient. Tave_{w} is a ratio of the test average value of the frequency points to the sample average value of the frequency points.

The step S104 comprises generating auditory gain values corresponding to the auditory compensation values according to the auditory compensation values, and enabling the user to enhance auditory sensitivity according to the auditory gain values.

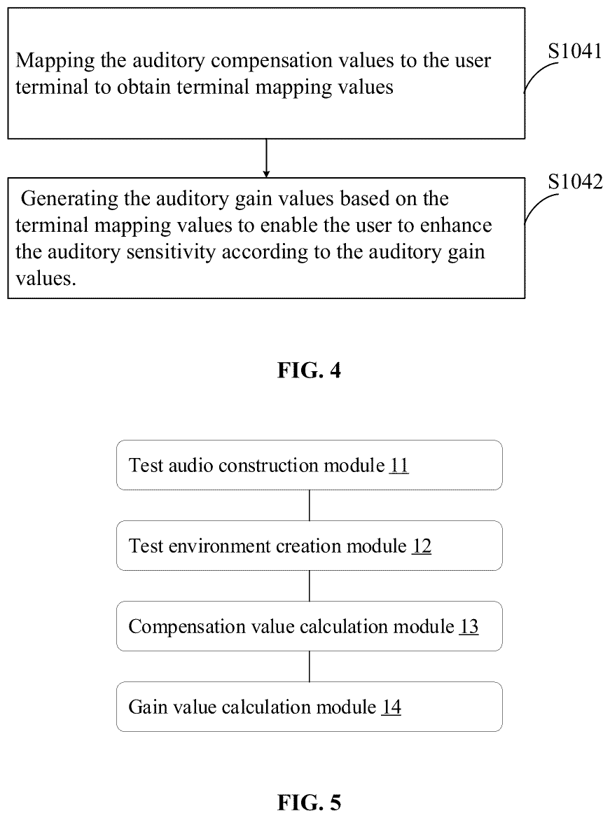

Furthermore, as shown in FIG. 4, the step S104 comprises Steps S1041-S1042.

The step S1041 comprises mapping the auditory compensation values to the user terminal to obtain terminal mapping values.

The step S1042 comprises generating the auditory gain values based on the terminal mapping values to enable the user to enhance the auditory sensitivity according to the auditory gain values.

In one specific embodiment, the auditory compensation values obtained are input into a user terminal (i.e., a terminal device with communication function such as the mobile phone), and are mapped into terminal mapping values through the user terminal. The terminal mapping values are transmitted to the pair of earphones, and the auditory gain values corresponding to the terminal mapping values are generated through filters of the pair of earphones, so that the user is able to use the pair of earphones and the auditory gain values to enhance corresponding frequency bands in audios to be played by the pair of earphones, thereby improving the auditory sensitivity of the user. Therefore, the user is able to hear the sound with the insensitive frequency bands and hear music in a wide frequency range.

In the auditory enhancement method of the present disclosure, the frequency points are extracted, the test short-time audio having different intensities is allocated to each of the frequency points, the auditory compensation values corresponding to the frequency points are obtained according to the test results fed back by the user and the identity information of the user, and the auditory gain values are generated corresponding to the auditory compensation values. Therefore, the user is enabled to enhance the auditory sensitivity according to the auditory gain values, thereby enhancing the perception intensity of the user on a sound with insensitive frequency bands without changing the sound volume of sound. Thus, the user is allowed to appreciate music in a wide frequency range.

### Embodiment 2

As shown in FIG. 5, the embodiment of the present disclosure provides an auditory enhancement system. The auditory enhancement system comprises a test audio construction module 11, a test environment creation module 12, a compensation value calculation module 13, and a gain value calculation module 14.

The test audio construction module 11 is configured to extract frequency points in initial audio data by a predetermined extraction rule and fuse an test short-time audio having different intensities to each of the frequency points to obtain test audio data.

The test environment creation module 12 is configured to create an auditory test environment for a user, play the test audio data to the user in the auditory test environment, and collect test results fed back by the user based on the test audio data.

Furthermore, the test environment creation module 12 comprises an environmental audio acquisition unit and a test environment creation unit.

The environmental audio acquisition unit is configured to obtain environmental audio data of a current environment and determine whether the environmental audio data exceeds an environmental audio threshold.

The test environment creation unit is configured to create the auditory test environment for the user based on the current environment when the environmental audio data does not exceed the environmental audio threshold.

The compensation value calculation module 13 is configured to obtain identity information of the user and input the identity information and the test results into an auditory test mathematical model to obtain auditory compensation values one-to-one corresponding to the frequency points in the test audio data.

Furthermore, the identity information comprises age information and gender information. The compensation value calculation module 13 comprises a difficulty analysis unit, a coefficient calculation unit, and a compensation value calculation unit.

The difficulty analysis unit is configured to perform difficulty analysis on the test short-time audio in each of the frequency points based on a difficulty database to obtain a difficulty coefficient corresponding to the test short-time audio in each of the frequency points. The coefficient calculation unit is configured to respectively calculate an age coefficient and a gender coefficient according to the age information and the gender information.

The compensation value calculation unit is configured to respectively calculate a weight value of each difficulty coefficient, a weight value of the age coefficient, a weight value of the gender coefficient, and an average value of the test results, and calculate the auditory compensation values respectively corresponding to the frequency points according to the weight value of each difficulty coefficient, the weight value of the age coefficient, the weight value of the gender coefficient, the average value of the test results, and the auditory test mathematical model.

The gain value calculation module 14 is configured to generate auditory gain values corresponding to the auditory compensation values according to the auditory compensation values, so that the user enhances auditory sensitivity according to the auditory gain values.

Furthermore, the gain value calculation module 14 comprises a compensation value mapping unit and a gain value calculation unit. The compensation value mapping unit is configured to map the auditory compensation values to the user terminal to obtain terminal mapping values. The gain value calculation unit is configured to generate the auditory gain values based on the terminal mapping values to enable the user to enhance the auditory sensitivity according to the auditory gain values.

Functions or operation steps implemented by the modules and units when they are executed are generally the same as the auditory enhancement method of Embodiment 1, which are not depicted in detail herein.

The auditory enhancement system in the embodiment of the present disclosure has the same implementation principle and technical effects as that in the Embodiment 1. For ease of description, for matters not mentioned in the auditory enhancement system, reference can be made to corresponding contents in the embodiment 1.

### Embodiment 3

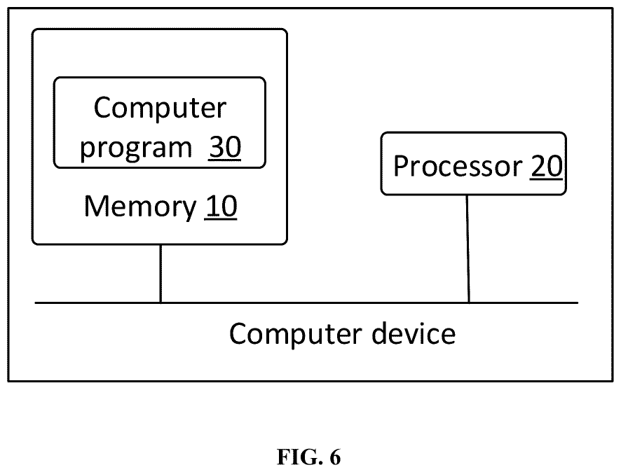

The present disclosure further provides a computer device. As shown in FIG. 6, the computer device comprises a memory 10, a processor 20, and a computer program 30. The computer program 30 is stored in the memory 10 and is executable on the processor 20. When the processor 20 executes the computer program 30, the auditory enhancement method is implemented.

The memory 10 comprises at least one type of storage medium, which comprises a flash memory, a hard disk, a multimedia card, a card-type memory (e.g., a solid disk or DX memory, etc.), a magnetic memory, a magnetic disk, an optical disk, etc. In some embodiments, the memory 10 may be an internal storage unit of the computer device, such as a hard disk of the computer device. In other embodiments, the memory 10 may be an external storage device, such as a plug-in hard disk, a smart memory card (SMC), a secure digital (SD) card, a flash card, etc. Further, the memory 10 may comprise both the internal storage unit of the computer device and the external storage device. The memory 10 is not only configured to store application software installed in the computer device and various types of data, but also to temporarily store data that has been output or is to be output.

In some embodiments, the processor 20 is an electronic control unit (ECU, also known as a vehicle computer), a central processing unit (CPU), a controller, a microcontroller, a microprocessor or other data processing chip. The processor 20 is configured to run program codes stored in the memory 10 or process data, such as executing an access restriction program.

It should be noted that structures shown in FIG. 6 do not constitute a limitation on the computer device. In other embodiments, the computer device may comprise fewer or more components than that shown in FIG. 6, the computer device may combine certain components or the components thereof are disposed in a different way.

The embodiment of the present disclosure further provides a readable storage medium on which a computer program is stored. When the computer program is executed by the processor, the auditory enhancement method as described above is implemented.

Those skilled in the art can understand that the logic and/or steps represented in the flowchart or described in other ways herein, for example, can be considered as a sequenced list of executable instructions for implementing logical functions, and can be specifically implemented in any computer-readable medium for use by an instruction execution system, device or equipment (such as a computer-based system, a system including a processor, or other system that is able to fetch instructions from an instruction execution system, device or equipment and execute instructions), or in combination with the instruction execution system, device or equipment. For the specification, “readable storage medium” may be any device that contains, stores, communicates, propagates or transmits a program for use by the instruction execution system, the device or the equipment, or in combination with the instruction execution system, the device or the equipment.

In one optional embodiment, the readable storage medium (a non-exhaustive list) includes an electrical connection portion (an electronic device) having one or more wiring, a portable computer disk cartridge (a magnetic device), a random access memory (RAM), a read-only memory (ROM), an erasable programmable read-only memory (EPROM or flash memory), a fiber optic device, and a compact disc read-only memory (CDROM). In addition, the readable storage medium may even be paper on which the computer program is printed or other suitable medium. The paper or other medium may be optically scanned, edited, interpreted, or otherwise processed in other suitable manners to obtain the computer program in an electronic manner, and then the computer program is stored in the memory of the computer device.

It should be understood that each part of the present disclosure may be implemented by hardware, software, firmware, or a combination thereof. In the above embodiments, steps or methods may be implemented by software or firmware stored in the memory and executed by the instruction execution system. For example, when being implemented in hardware, as in other embodiments, any of the following techniques known in the art, or a combination thereof, may be implemented: discrete logic circuits having logic gate circuits for implementing logic functions on data signals, application specific integrated circuits having suitable combinational logic gate circuits, programmable gate arrays (PGA), field programmable gate arrays (FPGAs), and the like.

Technical features of the above-mentioned embodiments can be combined arbitrarily. For the sake of brevity, all possible combinations of the technical features in the above-mentioned embodiments are not described. However, as long as there is no contradiction between the combinations of these technical features, the combinations should be considered to be within the scope of the specification.

The above-mentioned embodiments only represent some embodiments of the present disclosure. The descriptions thereof are specific and detailed, but should not be construed as a limitation of the scope of the present disclosure. It should be pointed out that for those of ordinary skill in the art, without departing from the concept of the present disclosure, modifications and improvements can be made. The modifications and the improvements belong to the protection scope of the present disclosure. Therefore, the protection scope of the present disclosure should be subject to the attached claims.

What is claimed is:

1. An auditory enhancement method, comprising steps:
extracting frequency points in initial audio data by a predetermined extraction rule, and fusing each of the frequency points with a test short-time audio having different intensities to obtain test audio data;
creating an auditory test environment for a user, playing the test audio data to the user in the auditory test environment, and collecting test results fed back by the user based on the test audio data, where the test results are respectively corresponding to the frequency points;
obtaining identity information of the user, and inputting the identity information and the test results into an auditory test mathematical model to obtain auditory compensation values one-to-one corresponding to the frequency points in the test audio data; and
generating auditory gain values corresponding to the auditory compensation values according to the auditory compensation values, and enabling the user to enhance auditory sensitivity according to the auditory gain values;
wherein the auditory enhancement method is applied to a pair of earphones, and the pair of earphones is communicated with a user terminal.

2. The auditory enhancement method according to claim 1, wherein the step of creating the auditory test environment for the user comprises steps:
obtaining environmental audio data of a current environment, and determining whether the environmental audio data exceeds an environmental audio threshold; and
creating the auditory test environment for the user based on the current environment when the environmental audio data does not exceed the environmental audio threshold.

3. The auditory enhancement method according to claim 1, wherein the identity information comprises age information and gender information, and the step of inputting the identity information and the test results into the auditory test mathematical model to obtain the auditory compensation values one-to-one corresponding to the frequency points in the test audio data comprises steps:
performing difficulty analysis on the test short-time audio at each of the frequency points based on a difficulty database to obtain a difficulty coefficient corresponding to the test short-time audio at each of the frequency points;
respectively calculating an age coefficient and a gender coefficient according to the age information and the gender information; and
respectively calculating a weight value of each difficulty coefficient, a weight value of the age coefficient, a weight value of the gender coefficient, and an average value of the test results, and calculating the auditory compensation values respectively corresponding to the frequency points according to the weight value of each difficulty coefficient, the weight value of the age coefficient, the weight value of the gender coefficient, the average value of the test results, and the auditory test mathematical model.

4. The auditory enhancement method according to claim 1, wherein the step of generating the auditory gain values corresponding to the auditory compensation values according to the auditory compensation values, and enabling the user to enhance the auditory sensitivity according to the auditory gain values comprises steps:
mapping the auditory compensation values to the user terminal to obtain terminal mapping values;
generating the auditory gain values based on the terminal mapping values to enable the user to enhance the auditory sensitivity according to the auditory gain values.

5. The auditory enhancement method according to claim 3, wherein expression formulas of each of the auditory compensation values are:
$$Tave = \frac{1}{N} * {\sum }_{i = 0}^{N - 1} ( {T}_{i} ) ;$$
$${Tave}_{W} = \frac{Tave}{{Tave}_{S}} ; and$$
$$S = B * ( Wt * D * {T}_{i} + Wa * A * {Tave}_{W} + Wg * G * {Tave}_{W} ) ;$$
where Tave represents the test average value of the test results of the frequency points, N represents the number of the frequency points, Tᵢ represents a test result of each of the frequency points, S represents a corresponding auditory compensation value, B represents an auditory compensation reference value, Wt represents the weight value of a sample average value of the frequency points, D represents a corresponding difficulty coefficient, A represents the age coefficient, Wa represents the weight value of the age coefficient, Taveₛ represents the sample average value of the frequency points, Tave_{w} represents a weight value of the test average value, Wa represents the weight value of the gender coefficient, and G represents the gender coefficient.

6. An auditory enhancement system, comprising:
a test audio construction module;
a test environment creation module;
a compensation value calculation module; and
a gain value calculation module;
wherein the test audio construction module is configured to extract frequency points in initial audio data by a predetermined extraction rule and fuse each of the frequency points with a short-time audio having different intensities to obtain test audio data;
the test environment creation module is configured to create an auditory test environment for a user, play the test audio data to the user in the auditory test environment, and collect test results fed back by the user based on the test audio data, and the test results are respectively corresponding to the frequency points;
the compensation value calculation module is configured to obtain identity information of the user and input the identity information and the test results into an auditory test mathematical model to obtain auditory compensation values one-to-one corresponding to the frequency points in the test audio data; and
the gain value calculation module is configured to generate auditory gain values corresponding to the auditory compensation values according to the auditory compensation values, so that the user enhances auditory sensitivity according to the auditory gain values.

7. The auditory enhancement system according to claim 6, wherein the test environment creation module comprises an environmental audio acquisition unit and a test environment creation unit;
wherein the environmental audio acquisition unit is configured to obtain environmental audio data of a current environment and determine whether the environmental audio data exceeds an environmental audio threshold; and
the test environment creation unit is configured to create the auditory test environment for the user based on the current environment when the environmental audio data does not exceed the environmental audio threshold.

8. The auditory enhancement system according to claim 6, wherein the identity information comprises age information and gender information, and the compensation value calculation module comprises a difficulty analysis unit, a coefficient calculation unit, and a compensation value calculation unit;
wherein the difficulty analysis unit is configured to perform difficulty analysis on the test short-time audio at each of the frequency points based on a difficulty database to obtain a difficulty coefficient corresponding to the test short-time audio at each of the frequency points;
the coefficient calculation unit is configured to respectively calculate an age coefficient and a gender coefficient according to the age information and the gender information; and
the compensation value calculation unit is configured to respectively calculate a weight value of each difficulty coefficient, a weight value of the age coefficient, a weight value of the gender coefficient, and an average value of the test results, and calculate the auditory compensation values respectively corresponding to the frequency points according to the weight value of each difficulty coefficient, the weight value of the age coefficient, the weight value of the gender coefficient, the average value of the test results, and the auditory test mathematical model.

9. A readable storage medium, comprising:
a computer program stored thereon;
wherein when the computer program is executed by a processor, the auditory enhancement method according to claim 1 is implemented.

10. A computer device, comprising:
a memory;
a processor; and
a computer program stored in the memory and executable on the processor;
wherein the processor is configured to execute a computer program to implement the auditory enhancement method according to claim 1.
